(19) United States
(12) Patent Application Publication
(10) Pub. No.: US 2024/0139292 A1
(43) Pub. Date: May 2, 2024

(54) COMPOSITIONS AND METHODS FOR TREATMENT OF SENSITIVE SKIN

(71) Applicant: PURE VITALITY, INC., Walnut Creek, CA (US)

(72) Inventor: Rachael Cabreira, Walnut Creek, CA (US)

(21) Appl. No.: 18/462,772

(22) Filed: Sep. 7, 2023

Related U.S. Application Data
(63) Continuation of application No. PCT/US2022/018551, filed on Mar. 2, 2022.
(60) Provisional application No. 63/158,041, filed on Mar. 8, 2021.

(51) Int. Cl. A61K 38/39; A61K 9/00; A61K 31/728; A61K 38/05; A61K 38/06; A61K 38/08; A61K 45/06; A61P 17/00

(52) U.S. Cl. CPC A61K 38/39; A61K 9/0014; A61K 31/728; A61K 38/05; A61K 38/06; A61K 38/08; A61K 45/06; A61P 17/00

(57) ABSTRACT

Compositions for treatment of sensitive skin comprising a peptide mixture, collagen, a first polyol and a second polyol are disclosed herein. Also disclosed are methods of using said compositions for increasing collagen levels in skin and improving or reducing discomfort of sensitive skin in a subject in need thereof.

### CROSS-REFERENCE TO RELATED APPLICATIONS

This application is a continuation of PCT Application No. PCT/US2022/018551, filed Mar. 2, 2022, which claims priority to and the benefit of U.S. Provisional Patent Application No. 63/158,041, filed Mar. 8, 2021, each of which are incorporated herein by reference. Any and all applications for which a foreign or domestic priority claim is identified in the Application Data Sheet as filed with the present application are hereby incorporated by reference under 37 C.F.R. § 1.57.

### FIELD OF THE DISCLOSURE

The present disclosure relates generally to the field of the treatment of sensitive skin. In particular, the disclosure relates to therapeutic compositions for use in increasing collagen levels in the skin to relieve, improve or reduce discomfort of sensitive skin. The present disclosure also relates to increasing collagen levels in sensitive skin of the genital area to improve sexual experience and well-being. The present disclosure also relates to use of the therapeutic compositions in conjunction with procedures associated with the skin to relieve, improve or reduce discomfort of sensitive skin.

### BACKGROUND

Skin diseases and disorders are ranked as the fourth most common cause of human illness, resulting in an enormous non-fatal burden. Skin diseases and disorders are accompanied with discomfort that can include stinging, burning and pain. Individuals having a skin disease or disorder associated with the sensitive skin of the genital area often experience decreased sexual experience; some are forced to avoid sexual activity as result of the discomfort. Increasing collagen levels in the skin can reduce the numerous unwanted effects of skin diseases and disorders. Particularly, increasing collagen levels in the skin of the genital area can increase the sexual experience of individuals suffering from skin diseases and disorders of the genital area. There exists a need for compositions and methods capable of increasing collagen levels in the skin.

### SUMMARY

The present disclosure is directed to therapeutic compositions for treatment of sensitive skin. Also provided herein are methods of using the therapeutic compositions to increase the amount of collagen in the skin of a subject in need thereof. Also provided herein are methods of using the therapeutic compositions to relieve, improve, or reduce discomfort of sensitive skin in a subject in need thereof. Some embodiments relate to treatment of sensitive skin of the genital area and to improving or enhancing sexual experience and well-being. Also provided herein are methods of making the therapeutic compositions.

Some embodiments provided herein relate to therapeutic compositions for treatment of sensitive skin. In some embodiments, the therapeutic compositions include a peptide mixture, collagen, a first polyol and a second polyol. In some embodiments, the peptide mixture comprises one or more of acetyl heptapeptide 4, azeloyl bis-dipeptide 10, copper peptide, heptapeptide 34, hexapeptide 70, palmitoyl tripeptide 1, palmitoyl tripeptide 5, palmitoyl tetrapeptide 7, trifluoroacetyl tripeptide 2, collagen, and hyaluronic acid. In some embodiments, the therapeutic compositions include: (a) from 0.3 to 5 weight percentage (% w/w) of the peptide mixture; (b) from 0.001 to 0.1% w/w collagen; (c) from 3 to 35% w/w of the first polyol; and (d) from 0.3 to 25% w/w of the second polyol. In some embodiments, weight percentage is based on a total weight of the therapeutic composition. In some embodiments, the therapeutic compositions include a combination of additives. In some embodiments, the combination of additives is one or more of an extract, a thickening agent, an antimicrobial agent, an anti-foaming agent, a surfactant, an antioxidant and a carboxylic acid. In some embodiments, the combination of additives is one or more of Zea mays oil, retinyl palmitate, chlolecaliferol, Echinacea purpurea extract, Ocimum sanctum extract, dextran, hydroxyethylcellulose, simethicone, polysorbate 20, triethanolamine, aminopropyl ascorbyl phosphate, phenoxylethanol, ethylhexylglycerin, caprylyl glycol, benzyl alcohol, citric acid, salicylic acid and sorbic acid. In some embodiments, the therapeutic composition has a pH level ranging from 2.4 to 6.4. In some embodiments, the therapeutic composition has a viscosity ranging from 2.0 to 4.0 pascal seconds (Pa·s). In some embodiments, the therapeutic composition has a density ranging from 0.93 to 1.09 grams per milliliter (g/mL). In some embodiments, the sensitive skin comprises the genital area. In some embodiments, the sensitive skin comprises vulvar tissue. In some embodiments, the therapeutic composition increases a collagen level of the sensitive skin.

Some embodiments provided herein relate to methods for increasing a collagen level in skin of a subject in need thereof. In some embodiments, the methods include selecting a subject. In some embodiments, the methods include topically applying to the skin a therapeutically effective amount of a therapeutic composition. In some embodiments, the methods include a therapeutic composition comprising a peptide mixture, collagen, a first polyol, and a second polyol. In some embodiments, the methods include the peptide mixture comprising one or more of acetyl heptapeptide 4, azeloyl bis-dipeptide 10, copper peptide, heptapeptide 34, hexapeptide 70, palmitoyl tripeptide 1, palmitoyl tripeptide 5, palmitoyl tetrapeptide 7, trifluoroacetyl tripeptide 2, collagen, and hyaluronic acid. In some embodiments, the methods include the therapeutic composition comprising: (a) from 0.3 to 5 weight percentage (% w/w) of the peptide mixture; (b) from 0.001 to 0.1% w/w collagen; (c) from 3 to 35% w/w of the first polyol; and (d) from 0.3 to 25% w/w of the second polyol. In some embodiments, weight percentage is based on a total weight of the therapeutic composition. In some embodiments, the methods relate to relieving, improving or reducing discomfort of the skin. In some embodiments, the methods relate to relieving, improving or reducing discomfort of the skin. In some embodiments, the discomfort is one or more of stinging, burning, pain, pruritus and tingling sensations. In some embodiments, the discomfort is accompanied by erythema. In some embodiments, the discomfort is associated with a skin condition. In some embodiments, the skin condition is a skin disorder or a skin wound. In some embodiments, the skin comprises sensitive skin of the genital area. In some embodiments, the sensitive skin comprises vulvar tissue. In some embodiments, the method improves or enhances sexual experience and sexual well-being.

These features, together with other features herein further explained, will be discussed in the following detailed description.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Embodiments provided herein relate to therapeutic compositions. Also provided are methods of making and using the therapeutic compositions.

### Compositions

Some embodiments provided herein relate to therapeutic compositions for treatment of sensitive skin. In some embodiments, the therapeutic compositions include a peptide mixture, collagen, a first polyol and a second polyol. As used herein, the term “composition” or “formulation” has its ordinary meaning in light of the specification, and refers to a combination of elements, components, or compositions presented together for a given purpose.

In some embodiments, the peptide mixture comprises two or more peptides. As used herein, the term “peptide” has its ordinary meaning when understood in light of the specification, and refers to polymeric structure of amino acid monomers. In some embodiments, the peptides comprise amino acid monomers that may be naturally occurring or synthetically produced. In some embodiments, the peptides may be chemically and/or semi-synthetically derivatized peptides. In some embodiments, the peptide mixture comprises one or more of acetyl heptapeptide 4, azeloyl bis-dipeptide 10, azeloyl bis-dipeptide 23, copper peptide (i.e., copper tripeptide), heptapeptide 32, heptapeptide 34, hexapeptide 70, palmitoyl tripeptide 1, palmitoyl tripeptide 5, palmitoyl tetrapeptide 7, trifluoroacetyl tripeptide 2, collagen, and hyaluronic acid. In some embodiments, the peptide mixture comprises azeloyl bis-dipeptide 10, copper peptide (i.e., copper tripeptide), heptapeptide 34, palmitoyl tripeptide 1, palmitoyl tripeptide 5, palmitoyl tetrapeptide 7, collagen, and hyaluronic acid. In some embodiments, the peptide mixture comprises one or more of azeloyl tetrapeptide 23, heptapeptide 32, palmitoyl tripeptide 5, palmitoyl tetrapeptide 7, collagen, and hyaluronic acid. In some embodiments, the peptide mixture comprises from about 0.5 parts per million (ppm) to about 100 ppm azeloyl bis-dipeptide 10, such as 0.5, 1, 2, 4, 6, 8, 10, 12, 20, 40, 60, 80, 100 ppm azeloyl bis-dipeptide 10, or a value within a range defined by any two of the aforementioned values. In some embodiments, the peptide mixture comprises from about 0.01 ppm to about 5 ppm copper peptide, such as 0.01, 0.05, 0.1, 0.15, 0.18, 0.2, 0.22, 0.24, 0.26, 0.28, 0.3, 0.4, 0.5, 0.6, 0.8, 1, 5 ppm copper peptide, or a value within a range defined by any two of the aforementioned values. In some embodiments, the peptide mixture comprises from about 0.01 ppm to about 5 ppm heptapeptide 34, such as 0.01, 0.03 0.05, 0.1, 0.15, 0.18, 0.2, 0.22, 0.24, 0.26, 0.28, 0.3, 0.4, 0.5, 0.6, 0.8, 1, 5 ppm heptapeptide 34, or a value within a range defined by any two of the aforementioned values. In some embodiments, the peptide mixture comprises from about 0.01 ppm to about 10 ppm palmitoyl tripeptide 1, such as 0.01, 0.05, 0.1, 0.2, 0.3, 0.4, 0.45, 0.47, 0.5, 0.52, 0.55, 0.6, 0.8, 1, 2, 5, 8, 10 ppm palmitoyl tripeptide 1, or a value within a range defined by any two of the aforementioned values. In some embodiments, the peptide mixture comprises from about 0.01 ppm to about 50 ppm palmitoyl tripeptide 5, such as 0.01, 0.1, 0.5, 1, 1.5, 2, 4, 6, 8, 10, 12, 20, 30, 40, 50 ppm palmitoyl tripeptide 5, or a value within a range defined by any two of the aforementioned values. In some embodiments, the peptide mixture comprises from about 0.0001 ppm to about 0.1 ppm palmitoyl tetrapeptide 7, such as 0.0001, 0.0005, 0.001, 0.0015, 0.002, 0.0022, 0.0024, 0.0026, 0.003, 0.004, 0.005, 0.001, 0.005, 0.01, 0.05, 0.1 ppm palmitoyl tetrapeptide 7, or a value within a range defined by any two of the aforementioned values.

In certain embodiments, the peptide mixture comprises collagen. The collagen may be a fibrillar collagen, a non-fibrillar collagen or both. In some embodiments, the collagen comprises Type I collagen, Type II collagen, Type III collagen, Type IV collagen, Type V collagen, Type XI collagen or combinations thereof. In certain some embodiments, the collagen comprises marine collagen. As used herein, the term “marine collagen” has its ordinary meaning when understood in light of the specification, and refers to a pure form of hypoallergenic protein produced from the skins of fish. Fish skins remaining from food production are thoroughly washed, then hydrolyzed by an acid and a food grade enzyme to yield marine collagen peptides, which have a low molecular weight for easy digestion and absorption. In some embodiments, the marine collagen is made from the scales of fresh, non-GMO, wild-caught fish. In some embodiments, the marine collagen comprises from about 60% w/w to about 95% w/w type I collagen, such as 60, 65, 70, 75, 80, 85, 90, 95% w/w type I collagen, or a value within a range defined by any two of the aforementioned values. In some embodiments, the marine collagen comprises proline, glycine, hydroxyproline and the combined amount of proline, glycine and hydroxyproline is from about 60% w/w to about 95% w/w, such as 60, 65, 70, 75, 80, 85, 90, 95% w/w, or in an amount within a range defined by any two of the aforementioned values. It will be appreciated that weight percentage is based on a total weight of the marine collagen. In some embodiments, the marine collagen has a water solubility, as measured from about 20° C. to about 40° C., ranging from about 0.1 milligrams per milliliter (mg/mL) to about 50 mg/mL, such as 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30, 35, 40, 45, 50 mg/mL, or a value within a range defined by any two of the aforementioned values. As described herein, “water solubility” has its ordinary meaning as understood in light of the specification, and refers to a gravimetric technique for measuring the maximum amount (mass) of solute that dissolves in an amount (mass) of solvent. In some embodiments, the peptide mixture comprises from about 0.01% w/w to about 5% w/w collagen, such as 0.01, 0.05, 0.1, 0.12, 0.14, 0.15, 0.16, 0.18, 0.2, 0.4, 0.6, 1, 2, 5% w/w collagen, or a value within a range defined by any two of the aforementioned values. In some embodiments, the peptide mixture comprises from about 0.01% w/w to about 5% w/w marine collagen, such as 0.01, 0.05, 0.1, 0.12, 0.14, 0.15, 0.16, 0.18, 0.2, 0.4, 0.6, 1, 2, 5% w/w marine collagen, or a value within a range defined by any two of the aforementioned values.

In some embodiments, the peptide mixture comprises hyaluronic acid. As used herein, the term “hyaluronic acid” is used interchangeably with the terms “hydrolyzed hyaluronic acid,” “sodium hyaluronate” and “hydrolyzed sodium hyaluronate.” In some embodiments, the peptide mixture comprises from about 0.01 ppm to about 50 ppm hyaluronic acid, such as 0.01, 0.1, 0.5, 1, 1.5, 2, 4, 6, 8, 10, 12, 20, 30, 40, 50 ppm hyaluronic acid, or a value within a range defined by any two of the aforementioned values.

In some embodiments, the peptide mixture comprises water, nonlimiting examples of which include distilled water, deionized water, purified water, a water previously processed in another manner, or a combination thereof. In some embodiments, the peptide mixture comprises from about 20% w/w to about 99% w/w water, such as 20, 30, 40, 50, 60, 70, 75, 80, 85, 86, 87, 88, 89, 90, 91, 92, 93, 94, 95, or 99% water, or a value within a range defined by any two of the aforementioned values.

In some embodiments, the therapeutic composition comprises from about 0.03% w/w to about 5% w/w peptide mixture, such as 0.03, 0.05, 0.1, 0.2, 0.5, 1.0, 1.1, 1.2, 1.3, 1.4, 1.5, 1.6, 1.7, 1.8, 1.9, 2, 2.5, 3, 3.5, 4, 4.5, 5% w/w peptide mixture, or a value within a range defined by any two of the aforementioned values.

In some embodiments, the therapeutic composition comprises collagen. The collagen of the therapeutic composition may be a collagen as described elsewhere herein. In some embodiments, the therapeutic composition comprises from about 0.0005% w/w to about 0.1% w/w collagen, such as 0.0005, 0.001, 0.0015, 0.0016, 0.0017, 0.0018, 0.0019, 0.002, 0.0021, 0.0022, 0.0023, 0.0024, 0.0025, 0.005, 0.01, 0.05, 0.1% w/w collagen, or a value within a range defined by any two of the aforementioned values. In some embodiments, the therapeutic composition comprises marine collagen. In some embodiments, the therapeutic composition comprises from about 0.0005% w/w to about 0.1% w/w marine collagen, such as 0.0005, 0.001, 0.0015, 0.0016, 0.0017, 0.0018, 0.0019, 0.002, 0.0021, 0.0022, 0.0023, 0.0024, 0.0025, 0.005, 0.01, 0.05, 0.1% w/w marine collagen, or a value within a range defined by any two of the aforementioned values.

In some embodiments, the therapeutic composition comprises a first polyol. As used herein, the term “polyol” has its ordinary meaning when understood in light of the specification, and refers to a chemical compound containing multiple hydroxyl groups (—OH groups). In some embodiments, the first polyol is a diol. As used herein, the term “diol” has its ordinary meaning when understood in light of the specification, and refers to a chemical compound containing two hydroxyl groups (—OH groups). In some embodiments, the diol is an aliphatic diol, an aromatic diol or a combination thereof. In some embodiments, the diol is a C₂-C₁₂ diol comprising a 1,2-diol, a 1,3-diol, 1,4-diol, 1,5-diol, a 1,6-diol or combinations thereof. In some embodiments, the diol is any isomer of ethanediol, propanediol, butanediol or combinations thereof. In several embodiments, the diol is selected from the group consisting of propanediol, caprylyl glycol, ethylhexylglycerin, or combinations of any of the foregoing. In some embodiments, the therapeutic composition comprises from about 3% w/w to about 35% w/w of the diol, such as 3, 5, 8, 10, 15, 15.1, 15.2, 15.3, 15.4, 15.5, 15.6, 15.7, 15.8, 15.9, 16, 20, 25, 20, 25, 30, 35% w/w of the diol, or a value within a range defined by any two of the aforementioned values. In several embodiments, the diol is propanediol. In some embodiments, the therapeutic composition comprises from about 3% w/w to about 35% w/w propanediol, such as 3, 5, 8, 10, 15, 15.1, 15.2, 15.3, 15.4, 15.5, 15.6, 15.7, 15.8, 15.9, 16, 20, 25, 20, 25, 30, 35% w/w propanediol, or a value within a range defined by any two of the aforementioned values.

In some embodiments, the therapeutic composition comprises a second polyol. In some embodiments, the second polyol is a triol containing three hydroxyl groups, a tetraol containing four hydroxyl groups, a pentaol containing five hydroxyl groups, a hexaol containing six hydroxyl groups or a combination thereof. In some embodiments, the second polyol is a C₂-C₁₂ aliphatic polyol, a C₂-C₁₂ aromatic polyol or a combination thereof. In several embodiments, the second polyol is selected from the group consisting of glycerin, triethanolamine, polysorbate 20, or combinations of any of the foregoing. In some embodiments, the therapeutic composition comprises from about 0.3% w/w to about 25% w/w of the second polyol, such as 0.3, 0.5, 1, 3, 5, 5.1, 5.2, 5.3, 5.4, 5.5, 5.6, 5.7, 5.8, 5.9, 6, 10, 15, 20, 25% w/w of the second polyol, or a value within a range defined by any two of the aforementioned values. In several embodiments, the second polyol is glycerin. In some embodiments, the therapeutic composition comprises from about 0.3% w/w to about 25% w/w glycerin, such as 0.3, 0.5, 1, 3, 5, 5.1, 5.2, 5.3, 5.4, 5.5, 5.6, 5.7, 5.8, 5.9, 6, 10, 15, 20, 25% w/w glycerin, or a value within a range defined by any two of the aforementioned values.

In some embodiments, the therapeutic composition comprises a combination of additives. Nonlimiting examples of additives of the therapeutic composition include an extract, a thickening agent, an antimicrobial agent, an anti-foaming agent, a surfactant, an antioxidant, a carboxylic acid, a peptide, or combinations of any of the foregoing. In some embodiments, the extract is an animal extract, a plant extract or a combination thereof. In some embodiments, the extract is Zea mays oil, retinyl palmitate, chlolecaliferol, Echinacea purpurea extract, Ocimum sanctum extract or a combination thereof. In some embodiments, the thickening agent is dextran, hydroxyethylcellulose, triglycerides, palmitates, myristates, stearates, polyethylene glycol, vegetable-based fatty alcohols, copolymers, cellulose gum, xanthan gum or a combination thereof. In some embodiments, the anti-foaming agent is simethicone. In some embodiments, the antimicrobial agent is caprylyl glycol, phenoxylethanol or a combination thereof. In some embodiments, the surfactant is a nonionic surfactant, an anionic surfactant or a combination thereof. In some embodiments, the surfactant is polysorbate 20. In some embodiments, the carboxylic acid is citric acid, salicylic acid, sorbic acid or a combination thereof. In some embodiments, the peptide is acetyl heptapeptide 4, azeloyl tetrapeptide 23, hexapeptide 70, heptapeptide 32, trifluoroacetyl tripeptide 2 or a combination thereof. In some embodiments, the antioxidant includes or more of the additives as described herein. In certain embodiments, the combination of additives is selected from the group consisting of aminopropyl ascorbyl phosphate, benzyl alcohol, caprylyl glycol, chlolecaliferol, citric acid, dextran, Echinacea purpurea extract, ethylhexylglycerin, hydroxyethylcellulose, Ocimum sanctum extract, phenoxylethanol, polysorbate 20, retinyl palmitate, triethanolamine, salicylic acid simethicone, sorbic acid, Zea mays oil, or combinations of any of the foregoing. In some embodiments, the therapeutic composition comprises from about 0.01% w/w to about 25% w/w additives, such as 0.01, 0.05, 0.1, 0.3, 0.5, 1, 3, 5, 5.1, 5.2, 5.3, 5.4, 5.5, 5.6, 5.7, 5.8, 5.9, 6, 10, 15, 20, 25% w/w additives, or a value within a range defined by any two of the aforementioned values.

In some embodiments, the therapeutic composition comprises water. The water of the therapeutic composition may be any water described herein. In some embodiments, the water is present in the therapeutic composition in an amount ranging from about from about 20% w/w to about 99% w/w, water, such as 20, 25, 30, 35, 40, 45, 50, 55, 60, 65, 66, 67, 68, 69, 70, 71, 72, 73, 74, 75, 80, 85, 90, 95, or 99% w/w water, or a value within a range defined by any two of the aforementioned values.

As used herein, the term “pH” has its ordinary meaning when understood in light of the specification, and refers to a concentration of hydronium ions, (i.e., level of acidity or basicity). In some embodiments, pH level is measured in accordance with test method USP 791 at 25° C. In some embodiments, the therapeutic composition provided herein has a pH level ranging from about 2.4 to about 6.4, such as 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, or a level within a range defined by any two of the aforementioned values.

As used herein, the term “viscosity” has its ordinary meaning when understood in light of the specification, and refers to a measure of a fluid's resistance to flow. In some embodiments, viscosity is measured in accordance with test method USP 912 using RVT Spindle #3 at 20 rpm, at 25° C. In some embodiments, the therapeutic composition provided herein has a viscosity ranging from about 0.5 pascal seconds (Pa·s) to about 10.0 Pa·s, such as 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, or a value within a range defined by any two of the aforementioned values.

As used herein, the term “density” has its ordinary meaning when understood in light of the specification, and refers to a measure of an object's mass per volume. In some embodiments, density is measured in accordance with test method USP 814, at 25° C. In some embodiments, the therapeutic composition provided herein has a density ranging from about 0.90 grams per milliliter (g/mL) to about 1.12 g/mL, such as 0.90, 0.92, 0.94, 0.96, 0.98, 1.00, 1.02, 1.04, 1.06, 1.08, 1.10, 1.12, or a value within a range defined by any two of the aforementioned values.

As used herein, the terms “microbe” and “microorganism” have their ordinary meaning when understood in light of the specification, and refer to microscopic organisms that comprise either a single cell (unicellular); cell clusters; or multicellular, relatively complex organisms. In some embodiments, microbe content is measured in accordance with test methods USP 61 and USP 62. In some embodiments, the therapeutic composition has a total microbe content of zero colony forming units per gram (cfu/g), i.e., the therapeutic composition is devoid of microbes. In some embodiments, the therapeutic composition has a yeast and mold content of zero cfu/g. In some embodiments, the therapeutic composition is devoid of coliform bacteria, Escherichia coli, Staphylococcus aureus, Pseudomonas aeruginosa or a combination thereof.

In some embodiments, the therapeutic composition is used for treatment of sensitive skin of the genital area. In some embodiments, the sensitive skin comprises vulvar tissue.

In some embodiments, the therapeutic composition increases a collagen level of the sensitive skin.

In several embodiments, the therapeutic composition comprises one or more pharmaceutically acceptable carriers, stabilizers, diluents, buffers, or components for application, storage, bioavailability, solubility, or other component parts that improve the efficacy, aesthetics, or other properties of the composition. “Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as topical, oral, or intravenous application, and administered in the form of a conventional pharmaceutical preparation, such as solid such as tablets, granules, powders, capsules, caplets, and liquid such as solution, spray, emulsion, foam, suspension, cream, lotion, ointment, salve, gel, and the like. Often the physiologically acceptable carrier is an aqueous pH buffered solution such as phosphate buffer or citrate buffer. The physiologically acceptable carrier may also include, for example, one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counter ions such as sodium, and nonionic surfactants such as Tween™, polyethylene glycol (PEG), and Pluronics™. Auxiliary, stabilizer, emulsifier, lubricant, binder, pH adjustor controller, isotonic agent, and other conventional additives may also be added to the carriers. The pharmaceutically acceptable or appropriate carrier may include other compounds known to be beneficial to an impaired situation of the skin, (e.g., antioxidants, such as Vitamin C, Vitamin E, Selenium or Zinc).

In certain embodiments, the therapeutic composition can be a formulated as a topical formulation. The topical formulation can further include, for example, a pharmaceutical vehicle that does not interfere with the function and viability of the peptide mixture, collagen, the first polyol and the second polyol. The “pharmaceutical vehicle” as described herein refers to an inert substance with which a medication is mixed to facilitate measurement and administration of the topical formulation.

In some embodiments, the peptide mixture, collagen, the first polyol and the second polyol can be used, for example, in the topical formulation including a pharmaceutically acceptable carrier prepared for storage and subsequent administration. As used herein, “topical” refers to the administration or application of a formulation to the skin or various body orifices. Some embodiments include use of the peptide mixture, collagen, the first polyol and the second polyol, as described herein, in combination with a pharmaceutically acceptable carrier or diluent. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives and stabilizers can be provided in the topical formulation. Preservatives can be used to keep the nutrients for the skin cells from breaking down. As used herein, the terms “carrier or diluent” may be a solid carrier or diluent for solid formulations, a liquid carrier or diluent for liquid formulations, or mixtures thereof. Solid carriers/diluents include, but are not limited to, a gum, a starch (e.g., cornstarch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. For liquid formulations, such as for topical or parenteral formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In addition, the topical formulation may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In some embodiments, the topical formulation including the peptide mixture, collagen, the first polyol and the second polyol can be formulated and used as a solution, spray, emulsion, foam, suspension, cream, lotion, ointment, salve, or gel for topical application. Suitable ingredients in the topical formulation can include a for example, water, saline, dextrose, mannitol, lactose, lecithin, albumin, or sodium glutamate, and the like. If desired, absorption enhancing preparations (for example, liposomes), can be utilized.

In some embodiments, the topical formulation can include at least one humectant that can be used for their moisturizing capabilities. Without being limiting, the humectant can include but are not limited to sodium PCA, nanolipid gels, glycerin, alpha-hydroxy acid, butylene glycol, propylene glycol, hexylene glycol, sorbitol, hyaluronic acid, urea, glyceryl triacetate, neoagarobiose, glycerol, xylitol, maltitol, polymeric polyols, polydextrose, quillaia, MP diol, seaweed and algae extracts, and lactic acid.

In some embodiments, the topical formulation further includes at least one preservative. Without being limiting, preservatives can include benzoin resin, jojoba, vitamin E, alcohol, phenoxyethanol, methylparaben, propylparaben, diazolidinyl urea, sorbic acid, and triclosan. In some embodiments, the at least one preservative is benzoin resin, jojoba, vitamin E, alcohol, phenoxyethanol, methylparaben, propylparaben, diazolidinyl urea, sorbic acid, and/or triclosan.

Coconut oil, olive oil, sesame oil, peanut oil, and soya can be used as suspension agents or lubricants in the topical formulation.

The topical formulation can further include, for example, one or more solvents, at least one botanical, and/or at least one emollient.

### Methods of Increasing Collagen

Embodiments provided herein relate to methods of increasing an amount of collagen. In some embodiments, the methods include increasing a collagen level in skin. In some embodiments, the methods include increasing a collagen level in the skin of a subject in need thereof. In some embodiments, the methods include a subject having levels of skin collagen that are diminished. In some embodiments, the methods include selecting a subject. In some embodiments, the subject is in need of an increased collagen level in the skin. In some embodiments, the methods include topically applying a therapeutic composition to the skin, a therapeutic composition as described elsewhere herein. In some embodiments, the therapeutic composition includes a peptide mixture, collagen, a first polyol and a second polyol, the peptide mixture, collagen, first polyol and second polyol, as described elsewhere herein.

In some embodiments, the methods include topically applying a therapeutically effective amount of the therapeutic composition to the skin. As used herein, the term “therapeutically effective amount” is an amount of the therapeutic composition that is capable of relieving, improving, or reducing discomfort of sensitive skin for which administration of the therapeutic composition is indicated. In some embodiments, the therapeutically effective amount ranges from about 0.01 mg/kg to about 400 mg/kg, such as 0.01, 0.05, 0.1, 0.5, 1, 2, 3, 4, 5, 10, 20, 30, 40, 50 60, 70, 80, 90, 100, 110, 120, 130, 140, 150, 160, 170, 180, 190, 200, or 400 mg/kg of the therapeutic composition, or an amount defined by any two of the aforementioned amounts. In some embodiments, the therapeutically effective amount ranges from about 10 mg to about 1000 mg, such as 10, 20, 30, 40, 50 60, 70, 80, 90, 100, 110, 120, 130, 140, 150, 200, 300, 500, 1000 mg of the therapeutic composition, or an amount defined by any two of the aforementioned amounts. In some embodiments, the therapeutic composition includes pharmaceutically acceptable carriers, stabilizers, diluents, buffers, or components for application, storage, bioavailability, solubility, or other component parts, or combinations thereof, that improve the efficacy, aesthetics, or other properties of the composition.

In some embodiments, the methods include topically applying the therapeutically effective amount of the therapeutic composition to the skin directly. In other words, no preparation of the therapeutic composition occurs before its use.

In some embodiments, the methods include preparing the therapeutic composition for use before topically applying the therapeutically effective amount of the composition to the skin. In some embodiments, the therapeutic composition is formed into an application preparation before topically applying the composition to the skin. In some embodiments, the application preparation includes the therapeutic composition and a portion of water or another solvent or liquid to be incorporated into the application preparation. In some embodiments, the application preparation is an emulsion. In some embodiments, the emulsion is selected from an oil-in-water emulsion, a water-in-oil emulsion, a water-in-oil-in-water emulsion, or combinations of any of the foregoing.

In some embodiments, the methods include topically applying the therapeutically effective amount of the therapeutic composition to the skin as a leave-on composition. The term “leave-on composition”, as used herein, is a composition that is applied to the skin without a deliberate rinsing step after application of the composition to the skin. In some embodiments, the methods include topically applying the therapeutically effective amount of the therapeutic composition to the skin as a wash-off composition. The term “wash-off composition”, as used herein, is a composition that is applied to the skin and subsequently washed away with a water rinse. In some embodiments, the wash-off composition is allowed to remain on the skin prior to being washed away for time period ranging from about 1 second (sec) to about 1 hour (h), such as 1 sec, 2 sec, 5 sec, 10 sec, 20 sec, 30 sec, 40 sec, 50 sec, 60 sec, 1.5 minutes (min), 3 min, 10 min, 20 min, 30 min, 40 min, 50 min, 1 h, or an amount within a range defined by any two of the aforementioned amounts.

In some embodiments, the methods include topically applying the therapeutically effective amount of the therapeutic composition to the skin one or more times per day. In some embodiments, the therapeutically effective amount of the therapeutic composition is topically applied to the skin 1, 2, 3, 4, 5, 6, 7, or 8 times per day. In some embodiments, the methods include topically applying the therapeutically effective amount of the therapeutic composition to the skin for one or more weeks. In some embodiments, the therapeutically effective amount of the therapeutic composition is topically applied to the skin for 1, 2, 3, 4, 5, 6, 7, 8, 9, 10, 11, 12, 13, 14, 15, 16 weeks. In some embodiments, the therapeutically effective amount of the therapeutic composition is topically applied to the skin at a weekly interval as desired by the subject in need thereof. In some embodiments, the therapeutically effective amount of the therapeutic composition is topically applied to the skin from a container or applicator and spread over and/or rubbed into the skin using the hands or fingers or an applicator device. In embodiments where the wash-off composition is applied to the skin a washing away step follows the step of topically applying the therapeutic composition.

In some embodiments, the methods comprise relieving, improving or reducing discomfort of the skin. The discomfort includes one or more of stinging, burning, pain, pruritus and tingling sensations. In some embodiments, the discomfort is accompanied by erythema. In some embodiments, the pain is derived from skin irritation, skin inflammation or a skin condition as described herein. In some embodiments, the inflammation is on the skin, vaginal area or the perineal area.

In some embodiments, the methods include discomfort that is associated with a skin condition. Without being limiting, the skin disorder can include skin diseases and skin damage. Nonlimiting examples of skin diseases include dermatitis, skin cancer (carcinoma and melanoma) and psoriasis. “Skin damage” as described herein, can refer to damage to the skin that can be caused by aging, sun damage, cancer, or skin diseases that can cause irritation of the skin. In some embodiments, the skin condition causes the subject to suffer, or otherwise experience discomfort. In some embodiments, the skin condition reduces, prevents, or otherwise interferes with the sexual experience of the subject, e.g., engaging in sexual activity. In some embodiments, the methods include discomfort that is associated with a skin wound.

In some embodiments, the methods include relieving, improving or reducing discomfort of sensitive skin of the genital area in a subject. The sensitive skin of the genital area includes vulvar tissue. As used herein, the term “vulvar tissue” refers to vulvar epithelium that is keratinized and nonkeratinized and includes the mons pubis and labia majora. In some embodiments, the methods improve or enhance the sexual experience and sexual well-being of the subject. In some embodiments, an improvement includes an enhancement of the sensory aspects of the superficial layers of the vulvar tissue. In some embodiments, an improvement includes increased sexual pleasure.

In some embodiments, the methods include increasing a collagen level in the skin. In some embodiments, the collagen level in the skin is increased from about 1% to about 50%, such as 1, 2, 5, 7, 10, 15, 20, 25, 30, 35, 40, 45, 50% or an amount within a range defined by any two of the aforementioned amounts.

### Use of Composition in Procedures

Embodiments provided herein relate to methods of performing a procedure associated with skin of a subject. The methods include selecting a subject in need of one or more of the procedures as described elsewhere herein. In some embodiments, the subject has previously experienced pain or discomfort while undergoing one or more procedures. The methods may include topically applying to the skin of the subject a therapeutically effective amount of the therapeutic composition. The methods may include passage of a period of time after topically applying the therapeutic composition. In some embodiments, the period of time is about 1, 2, 3, 4, 10, 12, 15, 20, 25, 30, 40, 60 minutes, or a period of time in between these values. The methods may include performing the procedure after the passage of the period of time.

In some embodiments of the method, the therapeutic composition includes a peptide mixture, collagen, a first polyol and/or a second polyol as described elsewhere herein. In some embodiments, the peptide mixture comprises one or more of acetyl heptapeptide 4, azeloyl bis-dipeptide 10, copper peptide, heptapeptide 34, hexapeptide 70, palmitoyl tripeptide 1, palmitoyl tripeptide 5, palmitoyl tetrapeptide 7, trifluoroacetyl tripeptide 2, collagen, and hyaluronic acid. In some embodiments, the therapeutic composition comprises: from 0.3 to 5 weight percentage (% w/w) of the peptide mixture; from 0.0005 to 0.1% w/w collagen; from 3 to 35% w/w of the first polyol; and from 0.3 to 25% w/w of the second polyol, wherein weight percentage is based on a total weight of the therapeutic composition.

In some embodiments, the procedure is selected from skin therapy, an examination, and any combination of the foregoing. In some embodiments, the procedure is skin therapy selected from anti-aging, cleansing, dermaplaning, eye massage, exfoliating, intense pulsed light (IPL), laser hair removal, amber light therapy, blue light therapy, infrared light therapy, red light therapy, lower-frequency pulsation, microcurrent technology, radiofrequency (RF) therapy, transdermal sonic (T-sonic) pulsation and any combination of the foregoing. In some embodiments, the procedure is selected from a diagnostic examination, a physical examination, and any combination of the foregoing. In some embodiments, the diagnostic examination includes an imaging method. A nonlimiting example of the imaging method is ultrasound (i.e., sonography). In some embodiments, the physical examination includes a pelvic examination.

In some embodiments, the method or procedure includes the use of a device. Nonlimiting examples of a device suitable for use include devices that are configured for dermaplaning, eye massage, exfoliating, intense pulsed light (IPL), laser hair removal, amber light therapy, blue light therapy, infrared light therapy, red light therapy, lower-frequency pulsation, microcurrent technology, radiofrequency (RF) therapy, and transdermal sonic (T-sonic) pulsation.

The terms “increasing” or “enhancing” as well as “reducing,” typically indicate a physiologically significant amount as determined by the subjective experience of the subject.

A “subject,” as used herein, includes any animal that exhibits a symptom, or is at risk for exhibiting a symptom, of sensitive skin of the genital area. Non-human primates and, preferably, human patients, are included.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

The components used to prepare the peptide mixture and the therapeutic composition disclosed herein are listed in Table 1.

TABLE 1

| | | | |
|---|---|---|---|
| | | Amount | Commercial |
| Trade Name | Description | (% w/w) | Source |

Peptide Mixture

| | | | |
|---|---|---|---|
| AC Marine | Soluble Collagen | 0.15 | Active |
| Collagen | 97.0-99.0% w/w | | Concepts |
| BiPure | Azelaoyl Bis- | 0.015 | Sinerga |
| | Dipeptide-10 | | |
| | 4.0-6.0% w/w | | |
| Citric Acid | Citric Acid, | 0.003 | Jungbunzlauer |
| | >99.5% w/w | | |
| Copper Peptide | Copper Tripeptide | 0.000025 | Skin Actives |
| GHK | >99.5% w/w | | |
| CytoRegulin | Heptapeptide-34 | 0.045 | Vantage |
| | 0.035-0.045% w/w | | |
| Matrixyl 3000 | Palmitoyl Tripeptide-1 | 0.045 | Sederma |
| | 0.090-0.110% w/w | | |
| | Palmitoyl Tetrapeptide-7 | | |
| | 0.004-0.006% w/w | | |
| Microcare SB | Sodium Benzoate | 0.015 | Thor |
| | 30% w/w | | |
| | Potassium Sorbate | | |
| | 15% w/w | | |
| MiniHA | Hydrolyzed Sodium | 0.00015 | Spectrum |
| | Hyaluronate | | |
| | >99.5% w/w | | |
| Syn-Coll | Palmitoyl Tripeptide-5 | 0.045 | DSM |
| | 0.40-0.60% w/w | | |

Therapeutic Composition

| | | | |
|---|---|---|---|
| Natrosol 250 | Hydroxyethylcellulose | 0.8 | Ashland |
| HHR CS | | | |
| Zemea | Propanediol | 8 | OMYA |
| Propanediol | | | |
| Glycerin | Glycerin | 2 | Norfox |
| | 99.7% w/w USP | | |
| Triethanolamine | Triethanolamine | 0.066 | Nexeo |
| | 99% w/w | | Solutions |
| Alkest TW 20 K | Polysorbate 20 | 0.5 | Norfox |
| Zemea | Propanediol | 5 | OMYA |
| Propanediol | | | |
| Holy Basil OSP | Propanediol; Ocimum | 0.5 | Biocogent |
| | Sanctum Extract | | |
| CytoCalm | Glycerin; Water; | 2 | Vantage |
| | Hexapeptide-70 | | Specialty |
| Fensebiome | Water; Acetyl | 1 | Lipotec USA |
| peptide solution | Heptapeptide-4; | | |
| | Caprylyl Glycol | | |
| Progeline | Glycerin; Water; | 1 | Lucas Meyer |
| | Dextran; Trifluoroacetyl | | Cosmetics |
| | Tripeptide-2 | | |
| Volpura EP | Echinacea Purpurea | 2 | Biocogent |
| | Extract; Propanediol | | |
| Vitamin A with | Retinyl Palmitate, Zea | 0.001 | The Personal |
| D3 | Mays (Corn) Oil; | | Formulator |
| | Chlolecaliferol | | |
| K3 Vita-C | Aminopropyl Ascorbyl | 0.1 | K3 Coporation |
| | Phosphate | | |
| Xiameter AFE- | Simethicone | 0.5 | Xiameter |
| 1520 Antifoam | | | |
| Emulsion | | | |
| Sharomix EG14 | Phenoxyethanol; | 1 | Sharon/ |
| | Ethylhexylglycerin | | Coastsouthwest |
| Citric Acid | Citric Acid | 0.001 | Jungbunzlauer |
| | 50% w/w Solution | | |

### Example 1

### Preparation of Peptide Composition

This example details the preparation of a peptide composition as disclosed herein.

A peptide composition was prepared by combining 45.0 milligrams (mg) AC Marine Collagen, 4.5 mg BiPure, 0.9 mg Citric Acid, 0.0072 mg Copper Peptide GHK, 13.5 mg CytoRegulin, 13.5 mg Matrixyl 3000, 4.5 mg Microcare SB, 0.045 mg MiniHA, 13.5 mg Syn-Coll and 26.8 mL water to form 30 g of the peptide composition.

### Example 2

### Preparation of Therapeutic Composition

This example details the preparation of a therapeutic composition as disclosed herein.

A volume of 345 mL deionized water was transferred to a compounding tank (i.e., “tank”) and vortex mixing was initiated. Natrosol 250 HHR CS (4 g), Zemea Propanediol (40 g), and Glycerin (10 g) were transferred to the tank during vortex mixing. Triethanolamine (033 g) was transferred to the tank portion-wise during vortex mixing to establish a pH level of the contents of the tank in a range of 8.0 to 8.5. Vortex mixing was continued for 20 minutes and the mixing rate was increased as the viscosity of the contents of the tank increased. Alkest TW 20 K (2.5 g), Zemea Propanediol (25 g), and Holy Basil OSP (2.5 g) were combined in a separate vessel with good mixing and transferred to the tank during vortex mixing. CytoCalm (10 g), Fensebiome peptide solution (5 g), Progeline (5 g), the peptide mixture prepared as described in Example 1 (7.5 g), Volpura EP (10 g), Vitamin A with D3 (5 mg), K3 Vita-C (0.5 g), were transferred to the tank during vortex mixing. Xiameter AFE-1520 Antifoam Emulsion (2.5 g) and deionized water (25 mL) were combined in a separate vessel with good mixing and transferred to the tank during vortex mixing. Sharomix EG14 (5 g) was transferred to the tank during vortex mixing. Citric acid (5 mg) was transferred to the tank portion-wise during vortex mixing to establish a pH level of the contents of the tank in a range of 3.8 to 4.0 and form 500 g of the therapeutic composition.

### Example 3

### Analysis of Therapeutic Composition

This example details the analysis of biological, chemical and physical properties of the therapeutic composition prepared as described in Example 2.

The pH level of the therapeutic composition was 3.90, as measured in accordance with test method USP 791, at 25° C. The viscosity of the therapeutic composition was a 2.6 pascal seconds (Pa·s), as measured in accordance with test method USP 912, using RVT Spindle #3 at 20 rpm, at 25° C. The density of the therapeutic composition was 1.029 grams per milliliter (g/mL), as measured in accordance with test method USP 814, at 25° C. The total microbe content of the therapeutic composition was zero colony forming units per gram (cfu/g), as measured in accordance with test method USP 61. The yeast and mold content of the therapeutic composition was a zero cfu/g, as measured in accordance with test method USP 61. The therapeutic composition was devoid of coliform bacteria, Escherichia coli, Staphylococcus aureus and Pseudomonas aeruginosa, as measured by USP 62.

### Example 4

### Use and Performance of Therapeutic Composition

This example details the use of the therapeutic composition prepared as described in Example 2.

Participants aged 30-65 years include seven females and five males. The therapeutic composition of Example 2 was applied topically to the skin in and around the vaginal area of each participant and/or of their sexual partner. Within 5 minutes after application of the therapeutic composition, the participants reported that discomfort of sensitive skin of the vaginal area was relieved, improved and/or reduced.

Within 5 minutes after application of the therapeutic composition, the participants engaged in sexual intercourse with their partner. None of the participants reported an adverse reaction as a result of using the therapeutic composition. Nearly all of the participants (83%) reported having an improved or enhanced sexual experience and well-being as a result of using the therapeutic composition. All of the participants reported a desire to continue use of the therapeutic composition.

All of the participants (100%) were highly pleased with the texture of the therapeutic composition and the feeling created by the therapeutic composition. Nearly all of the participants (92%) were highly pleased with the color, consistency and smell of the therapeutic composition.

### Example 5

### Use of the Therapeutic Composition to Increase Collagen Levels in the Skin

This prophetic example details the use of the therapeutic composition prepared as described in Example 2 to increase collagen levels in the skin.

Participants in the study are aged 30-65 years. The therapeutic composition (100-160 mg) of Example 2 is applied topically to the sensitive skin three times a day for a period of four weeks. After one week of application, at least 60% of the participants report less discomfort and improved appearance of the sensitive skin. After four weeks of application, at least 90% of the participants report less discomfort and improved appearance of the sensitive skin.

### Example 6

### Use of the Therapeutic Composition with Light Therapy

This prophetic example details the use of the therapeutic composition prepared as described in Example 2 in conjunction with a light therapy procedure.

Participants in the study are aged 30-65 years and had previously experienced pain or discomfort while undergoing a light therapy procedure. The therapeutic composition (100-160 mg) of Example 2 is applied topically to the skin of the participants. After a period of 5-15 minutes, the participants receive amber light therapy, blue light therapy, infrared light therapy, or red light therapy. At least 60% of the participants reported less pain or discomfort during the light therapy procedure and/or an improved outcome of the light therapy procedure based on the appearance of the skin. After one week, at least 90% of the participants reported less pain or discomfort during the light therapy procedure and/or an improved outcome of the light therapy procedure based on the appearance of the skin as a result of the light therapy procedure.

### Example 7

### Use of the Therapeutic Composition with a Dermaplaning Device

This prophetic example details the use of the therapeutic composition prepared as described in Example 2 in conjunction with a dermaplaning device.

Participants in the study are aged 30-65 years and had previously experienced pain or discomfort while undergoing a procedure with a dermaplaning device. The therapeutic composition (100-160 mg) of Example 2 is applied topically to the skin of the participants. After a period of 5-15 minutes, the skin of the participants is treated with a dermaplaning device. At least 60% of the participants reported less pain or discomfort during the treatment and/or an improved outcome of the treatment based on the appearance of the skin. After one week, at least 90% of the participants reported less pain or discomfort during the treatment and/or an improved outcome of the treatment based on the appearance of the skin as a result of the dermaplaning device treatment.

### Example 8

### Use of the Therapeutic Composition with Radiofrequency (RF) Therapy

This prophetic example details the use of the therapeutic composition prepared as described in Example 2 in conjunction with radiofrequency (RF) therapy.

Participants in the study are aged 30-65 years and had previously experienced pain or discomfort while undergoing a radiofrequency (RF) therapy procedure. The therapeutic composition (100-160 mg) of Example 2 is applied topically to the skin of the participants. After a period of 5-15 minutes, the participants receive radiofrequency (RF) therapy. At least 60% of the participants reported less pain or discomfort during the radiofrequency (RF) therapy procedure and/or an improved outcome of the radiofrequency (RF) therapy procedure based on the appearance of the skin. After one week, at least 90% of the participants reported less pain or discomfort as a result of the radiofrequency (RF) therapy procedure.

### Example 9

### Use of the Therapeutic Composition with Ultrasound

This prophetic example details the use of the therapeutic composition prepared as described in Example 2 in conjunction with an ultrasound procedure.

Participants in the study are aged 30-65 years and had previously experienced pain or discomfort while undergoing an ultrasound procedure. The therapeutic composition (100-160 mg) of Example 2 is applied topically to the skin of the participants. After a period of 5-15 minutes, the participants undergo an ultrasound procedure. At least 60% of the participants reported less pain or discomfort during the ultrasound procedure. After one week, at least 90% of the participants reported less pain or discomfort as a result of the ultrasound procedure.

### Example 10

### Use of the Therapeutic Composition with Pelvic Examination

This prophetic example details the use of the therapeutic composition prepared as described in Example 2 in conjunction with a pelvic examination.

Participants in the study are aged 30-65 years and had previously experienced pain or discomfort while undergoing a pelvic examination. The therapeutic composition (100-160 mg) of Example 2 is applied topically to the skin of the participants. After a period of 5-15 minutes, the participants undergo a pelvic examination. At least 60% of the participants reported less pain or discomfort during the pelvic examination. After one week, at least 90% of the participants reported less pain or discomfort as a result of the pelvic examination.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

Unless defined otherwise, technical and scientific terms used herein have the same meaning as commonly understood by one of ordinary skill in the art to which the present disclosure belongs. All patents, applications, published applications and other publications referenced herein are expressly incorporated by reference in their entireties unless stated otherwise. For purposes of the present disclosure, the following terms are defined below.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A therapeutic composition for treatment of sensitive skin comprising:
a peptide mixture;
collagen;
a first polyol; and
a second polyol.

2. The therapeutic composition of claim 1, wherein the peptide mixture comprises one or more of acetyl heptapeptide 4, azeloyl bis-dipeptide 10, copper peptide, heptapeptide 34, hexapeptide 70, palmitoyl tripeptide 1, palmitoyl tripeptide 5, palmitoyl tetrapeptide 7, trifluoroacetyl tripeptide 2, collagen, and hyaluronic acid.

3. The therapeutic composition of claim 1, wherein the composition comprises:
from about 0.3 to 5 weight percentage (% w/w) of the peptide mixture;
from about 0.0005 to 0.1% w/w collagen;
from about 3 to 35% w/w of the first polyol; and
from about 0.3 to 25% w/w of the second polyol,
wherein weight percentage is based on a total weight of the composition.

4. The therapeutic composition of claim 1, wherein the composition further comprises a combination of additives.

5. The therapeutic composition of claim 1, wherein the combination of additives is one or more of an extract, a thickening agent, an antimicrobial agent, an anti-foaming agent, a surfactant, an antioxidant a peptide, and a carboxylic acid.

6. The therapeutic composition of claim 1, wherein the combination of additives is one or more of Zea mays oil, retinyl palmitate, chlolecaliferol, Echinacea purpurea extract, Ocimum sanctum extract, dextran, hydroxyethylcellulose, simethicone, polysorbate 20, triethanolamine, aminopropyl ascorbyl phosphate, phenoxylethanol, ethylhexylglycerin, caprylyl glycol, benzyl alcohol, citric acid, salicylic acid and sorbic acid.

7. The therapeutic composition of claim 1, wherein the composition has a pH level ranging from 2.4 to 6.4.

8. The therapeutic composition of claim 1, wherein the composition has a viscosity ranging from 2.0 to 4.0 pascal seconds (Pa·s).

9. The therapeutic composition of claim 1, wherein the composition has a density ranging from 0.93 to 1.09 grams per milliliter (g/mL).

10. The therapeutic composition of claim 1, wherein the sensitive skin comprises the genital area.

11. The therapeutic composition of claim 1, wherein the sensitive skin comprises vulvar tissue.

12. The therapeutic composition of claim 1, wherein the composition increases a collagen level of the sensitive skin.

13. A method for increasing a collagen level in skin of a subject, the method comprising:
selecting the subject; and
topically applying to the skin a therapeutically effective amount of a therapeutic composition, the therapeutic composition comprising:
a peptide mixture,
collagen,
a first polyol, and
a second polyol.

14. The method of claim 13, wherein the peptide mixture comprises one or more of acetyl heptapeptide 4, azeloyl bis-dipeptide 10, copper peptide, heptapeptide 34, hexapeptide 70, palmitoyl tripeptide 1, palmitoyl tripeptide 5, palmitoyl tetrapeptide 7, trifluoroacetyl tripeptide 2, collagen, and hyaluronic acid.

15. The method of claim 13, wherein the therapeutic composition comprises:
from about 0.3 to 5 weight percentage (% w/w) of the peptide mixture;
from about 0.001 to 0.1% w/w collagen;
from about 3 to 35% w/w of the first polyol; and
from about 0.3 to 25% w/w of the second polyol,
wherein weight percentage is based on a total weight of the composition.

16. The method of claim 13, wherein the method comprises relieving, improving or reducing discomfort of the skin.

17-23. (canceled)

24. A method of performing a procedure associated with skin of a subject, the method comprising:
selecting the subject;
topically applying a therapeutic composition to the skin of the subject; and
performing the procedure,
wherein the therapeutic composition comprises:
a peptide mixture,
collagen,
a first polyol, and
a second polyol.

25. The method of claim 24, wherein the peptide mixture comprises one or more of acetyl heptapeptide 4, azeloyl bis-dipeptide 10, copper peptide, heptapeptide 34, hexapeptide 70, palmitoyl tripeptide 1, palmitoyl tripeptide 5, palmitoyl tetrapeptide 7, trifluoroacetyl tripeptide 2, collagen, and hyaluronic acid.

26. The method of claim 24, wherein the therapeutic composition comprises:
from about 0.3 to 5 weight percentage (% w/w) of the peptide mixture;
from about 0.0005 to 0.1% w/w collagen;
from about 3 to 35% w/w of the first polyol; and
from about 0.3 to 25% w/w of the second polyol,
wherein weight percentage is based on a total weight of the therapeutic composition.

27. The method of claim 24, wherein the procedure is selected from skin therapy, an examination, and any combination of the foregoing.

28-30. (canceled)
